(19) United States
(12) Patent Application Publication
(10) Pub. No.: US 2020/0016437 A1
(43) Pub. Date: Jan. 16, 2020

(54) Skin Care Compositions

(71) Applicant: Melaleuca, Inc., Idaho Falls, ID (US)

(72) Inventor: Dzung Le, Idaho Falls, ID (US)

(73) Assignee: Melaleuca, Inc., Idaho Falls, ID (US)

(21) Appl. No.: 16/277,571

(22) Filed: Feb. 15, 2019

Related U.S. Application Data
(63) Continuation of application No. 15/265,351, filed on Sep. 14, 2016, now abandoned.
(60) Provisional application No. 62/218,316, filed on Sep. 14, 2015.

(51) Int. Cl. A61Q 19/10; A61K 8/9789; A61Q 19/08; A61K 8/99; A61Q 1/14; A61K 8/49; A61Q 19/00

(52) U.S. Cl. CPC A61Q 19/10; A61K 8/9789; A61Q 19/08; A61K 2800/5922; A61Q 1/14; A61K 8/4946; A61Q 19/007; A61K 8/99

(57) ABSTRACT

This document provides skin care compositions that can include one or more saponin extracts. In some cases, the skin care compositions provided herein can include a saponin extract (e.g., a Sapindus mukorossi extract). In some cases, the skin care compositions provided herein can include a Sapindus mukorossi extract, allantoin, carnosine, and a plant extract blend. The plant extract blend can include bioflavonoids (e.g., citrus bioflavonoids), a Brassica oleracea ver. italica (i.e., broccoli) extract, and an Aloe barbadensis leaf extract, or combinations thereof.

### CROSS-REFERENCE TO RELATED APPLICATIONS

This application is a continuation of U.S. application Ser. No. 15/265,351, filed Sep. 14, 2016, which claims priority to U.S. Provisional Patent Application No. 62/218,316, filed Sep. 14, 2015, entitled “Skin Care Compositions,” which are incorporated by reference herein in their entirety.

### TECHNICAL FIELD

This document relates to the field of skin care compositions. For example, this document relates to skin care compositions for improving the condition and appearance of human or animal skin.

### BACKGROUND INFORMATION

Many people desire improving the health and physical appearance of their skin. In many cases, people desire to improve the health and physical appearance of their skin by delaying, reducing or eliminating environmental damage or histological changes associated with aging, such as wrinkling, to human skin by improving the aesthetical characteristics of the skin through minimizing, or even eliminating, the appearance of wrinkles, pores, and uneven skin color and texture. Accordingly, there is a continuing need to formulate skin care compositions that improve the health and physical appearance of skin.

### SUMMARY

This document provides skin care compositions. For example, this document provides methods and materials (e.g., skin care compositions) for improving the condition and appearance of human or animal skin. In some cases, the skin care compositions provided herein can include a saponin extract (e.g., a Sapindus mukorossi extract). In some cases, the skin care compositions provided herein can include a Sapindus mukorossi extract, allantoin, carnosine, and a plant extract blend. In some cases, the plant extract blend can contain bioflavonoids (e.g., citrus bioflavonoids), a Brassica oleracea ver. italica extract, and an Aloe barbadensis leaf extract, or combinations thereof.

In general, one aspect of this document features a skin care composition comprising (a) a Sapindus mukorossi extract in an amount from about 0.001% to about 0.5% by weight of said composition, (b) allantoin in an amount from about 0.05% to about 1% by weight of said composition, and (d) carnosine from about 0.1% to about 2% by weight of said composition. The composition can further comprises a plant extract blend in an amount from about 0.1% to about 10% by weight of said composition, wherein the plant extract blend comprises a bioflavonoid, a Brassica oleracea ver. italica extract, and an Aloe barbadensis leaf extract. The composition provided herein can optionally comprise a plankton extract from about 0.1% to about 5% by weight of said composition. The composition can be a topical skin care formulation. In some cases, the application of the composition to human skin causes the skin to become hydrated. In some cases, the application of the composition to human skin causes the skin to be cleansed of makeup, dirt and excess oil.

Unless otherwise defined, all technical and scientific terms used herein have the same meaning as commonly understood by one of ordinary skill in the art to which this invention pertains. Although methods and materials similar or equivalent to those described herein can be used to practice the invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

The details of one or more embodiments of the invention are set forth in the accompanying drawings and the description below. Other features, objects, and advantages of the invention will be apparent from the description and drawings, and from the claims.

### DETAILED DESCRIPTION

This document provides skin care compositions that can include one or more saponin extracts. In some cases, the skin care compositions provided herein can include a Sapindus mukorossi extract. In some cases, the skin care compositions provided herein can include a Sapindus mukorossi extract, allantoin, carnosine, a plant extract blend containing bioflavonoids (e.g., citrus bioflavonoids), a Brassica oleracea ver. italica (i.e., broccoli) extract, and an Aloe barbadensis leaf extract, or combinations thereof. For example, a skin care composition provided herein can include a Sapindus mukorossi extract, allantoin, carnosine, and a plant extract blend. In some cases, the plant blend extract can contain bioflavonoids (e.g., citrus bioflavonoids), a Brassica oleracea ver. italica extract, and an Aloe barbadensis leaf extract, or combinations thereof. The composition provided herein can optionally comprise a plankton extract. In some cases, the skin care compositions provided herein can be a topical skin care formulation.

As described herein, a skin care composition provided herein can include a saponin extract obtained from a plant source. Suitable plant sources of a saponin extract can include, without limitation, shikakai, soyabeans, beans, peas (Pisum sativum), lucerne, tea, spinach, sugar beet, quinoa, liquorice, sunflower, horse chestnut, ginseng, oats, capsicum peppers, aubergine, tomato seed, alliums, asparagus, yam, fenugreek, yucca and ginseng, lucerne, mung beans, Bupleurum falcatum, Camellia oleifera, Camellia sinensis Desmodium adscendens, Gypsophila, Panax quinqufolius, Panax japonicas, Quillaja saponaria, Sapindus delavayi, Sapindus marginatus, Sapindus saponaria, Sapindus trifoliatus, Saponaria officinalis, Styrax japonica, Yucca schidigera or combinations thereof. In some cases, any appropriate part of the plant source can be used for extracting saponin material, including leaves, stems, roots, bulbs, blossom and fruit (including the skin, flesh, and/or seed of the fruit). One example of a saponin extract that can be used to make a skin care composition provided herein includes a saponin extract from a Sapindus mukorossi tree. In some cases, a Sapindus mukorossi extract can include saponin compounds of triterpene saponin glucosides.

Sapindus mukorossi extract can be obtained as described elsewhere (e.g., U.S. Patent Application Publication No. 2013/0295204). In some cases, a Sapindus mukorossi extract can be obtained commercially. For example, Sapindus mukorossi extracts can be obtained from Barnet Products Corporation (Englewood Cliffs, N.J.; Barcleanse NL).

In some cases, a skin care composition can contain one or more than one Sapindus mukorossi extract. A skin care composition provided herein can contain any appropriate amount of a Sapindus mukorossi extract. For example, a skin care composition provided herein can contain from about 0.001% to about 3% by weight (w/w) (e.g., from about 0.001% to about 2% w/w, from about 0.001% to about 0.5% w/w, from about 0.001% to about 0.005% w/w, from about 0.005% to about 0.01% w/w, from about 0.01% to about 0.5% w/w, from about 0.01% to about 0.05% w/w, from about 0.05% to about 0.1% w/w, from about 0.1% to about 0.5% w/w, from about 0.5% to about 1% w/w, or from about 1% to about 3% w/w) of a Sapindus mukorossi extract. In some cases, at least 0.001% w/w (e.g., at least 0.001% w/w, 0.002% w/w, 0.003% w/w, 0.004% w/w, 0.005% w/w, 0.01% w/w, 0.05% w/w, 0.1% w/w, 0.5% w/w, 1% w/w, 2% w/w, or 3% w/w) of a skin care composition provided herein can be a Sapindus mukorossi extract.

In some cases, a skin care composition provided herein can include, without limitation, allantoin (which can also be referred to as 5-ureidohydantoin, glyoxyldiureide, 5-ureidohydantoin, and 2,5-dioxo-4-imidazolidinyl urea). Allantoin is a product of oxidation of uric acid, and its chemical formula is C₄H₆N₄O₃. Allantoin is a botanical extract of the comfrey plant that, in some cases, can be used to make a skin care composition provided herein.

Allantoin can be prepared as described elsewhere (e.g., U.S. Pat. No. 5,196,545). In some cases, allantoin can be obtained commercially. For example, allantoin can be obtained from 3V Incorporated (Georgetown, S.C.).

In some cases, a skin care composition provided herein can contain one or more than one allantoin. A skin care composition provided herein can contain any appropriate amount of allantoin. In some cases, a skin care composition provided herein can contain from about 0.01% to about 5% by weight (w/w) (e.g., from about 0.01% to about 0.05% w/w, from about 0.05% to about 0.1% w/w, from about 0.1% to about 0.5% w/w, from about 0.5% to about 1% w/w, from about 1% to about 3% w/w, or from about 3% to about 5% w/w) of allantoin. For example, at least 0.01% w/w (e.g., at least 0.01% w/w, 0.05% w/w, 0.1% w/w, 0.5% w/w, 1% w/w, 2% w/w, 3% w/w, 4% w/w, or 5% w/w) of a skin care composition provided herein can be allantoin.

In some cases, a skin care composition provided herein can optionally include, without limitation, a plankton extract. Plankton extracts, such as a marine plankton extract, can be obtained as a marine exopolysaccharide secretion from a planktonic micro-organism. In some cases, a plankton extract used to make a skin care composition provided herein can include a polysaccharide composed of galactose and N-acetyl-glucosamine units. Examples of planktons that can be used to make plankton extracts used to make a skin care composition provided herein include, without limitation, thalasso plankton, green micro-algae, diatoms, greenish-blue, and nitrogen-fixing seaweed.

Plankton extracts can be obtained using appropriate extraction techniques. In some cases, a plankton extract can be obtained commercially. For example, marine plankton extracts can be obtained from Barnet Products Corporation (Englewood Cliffs, N.J.; Benoitine).

In some cases, a skin care composition can contain one or more than one plankton extract. A skin care composition provided herein can contain any appropriate amount of a plankton extract (e.g., a marine plankton extract). For example, a skin care composition provided herein can contain from about 0.01% to about 5% by weight (w/w) (e.g., from about 0.01% to about 0.05% w/w, from about 0.05% to about 0.1% w/w, from about 0.1% to about 0.5% w/w, from about 0.5% to about 1% w/w, from about 1% to about 2% w/w, from about 2% w/w to about 3% w/w, or from about 3% to about 5% w/w) of a plankton extract. In some cases, at least 0.001% w/w (e.g., at least 0.01% w/w, 0.05% w/w, 0.1% w/w, 0.5% w/w, 1% w/w, 2% w/w, 3% w/w, 4% w/w, or 5% w/w) of a skin care composition provided herein can be composed of a plankton extract.

In some cases, a skin care composition provided herein can include one or more than one carnosine, which can also be referred to as B-alanyl-L-histidine, or may be known as Dragosine. Carnosine is a zwitterionic dipeptide of amino acids beta-alanine and histidine.

Carnosine can be prepared as described elsewhere (e.g., U.S. Pat. No. 4,359,416). In some cases, carnosine can be obtained commercially. For example, carnosine can be obtained from Symrise (Holzminden, Germany; Dragosine® P.N. 844033).

In some cases, a skin care composition provided herein can contain one or more than one carnosine. A skin care composition provided herein can contain any appropriate amount of carnosine. For example, at least 0.01% w/w (e.g., at least 0.01% w/w, 0.05% w/w, 0.10% w/w, 0.15% w/w, 0.50% w/w, 1% w/w, 2% w/w, 3% w/w, 4% w/w, or 5% w/w) of a skin care composition provided herein can be composed of a carnosine. In some embodiments, a skin care composition provided herein can contain carnosine from about 0.01% to about 5% by weight (w/w) (e.g., from about 0.01% to about 0.05% w/w, from about 0.05% to about 0.1% w/w, from about 0.1% to about 0.15% w/w, from about 0.15% to about 0.2% w/w, from about 0.2% to about 0.5% w/w, from about 0.5% to about 1% w/w, from about 1% to about 3% w/w, or from about 3% to about 5% w/w).

As described herein, a skin care composition provided herein can include plant extract blends containing two or more (e.g., two or three) of the following ingredients: bioflavonoids (e.g., citrus bioflavonoids), Brassica oleracea ver. italica (i.e., broccoli) extracts, and Aloe barbadensis leaf extracts.

The plant extract blends provided herein can be prepared using appropriate extraction techniques. In some cases, a plant extract blend provided herein can be obtained commercially. For example, a plant extract blend that includes bioflavonoids, a Brassica oleracea ver. italica extract, and an Aloe barbadensis leaf extract can be obtained from BioSpectrum, Inc. (Sangdaewon-dong, Korea; BioDtox™).

In some cases, a skin care composition provided herein can contain one or more than one plant extract blend that includes bioflavonoids, Brassica oleracea ver. italica extracts, and Aloe barbadensis leaf extracts. A skin care composition provided herein can contain any appropriate amount of a plant extract blend. For example, at least 0.1% w/w (e.g., at least 0.1% w/w, 0.5% w/w, 1% w/w, 2% w/w, 3% w/w, 4% w/w, 5% w/w, 6% w/w, 7% w/w, 8% w/w, 9% w/w, or 10% w/w) of a skin care composition provided herein can be composed of a plant extract blend provided herein. Some embodiments of a skin care composition provided herein can contain a plant extract blend from about 0.01% to about 5% by weight (w/w) (e.g., from about 0.01% to about 0.015% w/w, from about 0.015% to about 0.02% w/w, from about 0.02% to about 0.05% w/w, from about 0.05% to about 0.1% w/w, from about 0.1% to about 0.5% w/w, from about 0.5% to about 1% w/w, from about 1% to about 3% w/w, or from about 3% to about 5% w/w, from about 5% to about 7% w/w, or from about 7% to about 10% w/w).

In some cases, a skin care composition provided herein can contain a variety of optional ingredients including, without limitation, water, witch hazel, 1,2-hexanediol, caprylyl glycol, propandiol, a zinc salt of 1-pyrrolidone carboxylate (i.e., zinc PCA), polysorbate 20, trehalose, bis-ethoxydiglycol cyclohexane 1,4-dicarboxylate, xanthan gum, disodium edta, betaine, a sodium salt of 1-pyrrolidone carboxylate (i.e., sodium PCS), sorbitol, serine, glycine, glutamic acid, alanine, lysine, arginine, threonine, proline, methylparaben, propylparaben, tetrapeptide-17, glycerin, butylene glycol, aqua, cucumis sativus (cucumber) fruit extract, citric acid, parfums/fragrances, hydrolyzed hyaluronic acid, a fomes officinalis (mushroom) extract, dimethicone/vinyl dimethicone crosspolymer, silica, aqua, decyl glucoside, sodium cocoyl isethionate, lauryl lactyl lactate, sodium methyl cocoyl taurate, sodium methyl-2 sulfolaurate, disodium 2-sulfolaurate, sodium lauryl sulfoacetate, sodium lactate, cocamidopropyl betaine, collodial oatmeal, cetyl alcohol, beta-glucan, glyceryl stearate, peg-100 stearate, stearic acid, sodium benzoate, a tremella fuciformis sporocarp (silver ear mushroom) extract, hydrogenated lecithin, polyquaternium-7, sodium methyl cocyl taurate, myristic acid, lauric acid, potassum hydroxide, disodium ethylenediaminetetraacetic acid (EDTA), potassium sorbate, niacinamide, calcium pantothenate, sodium ascorbyl phosphate, tocopheryl acetate, pyridoxine hydrochloride (HCL), maltodextrin, sodium starch octenylsuccinate, silica, montmorillonite, c12-16 alcohols, palmitic acid, lysolecithin, sclerotium gum, pullulan, sodium glutamate, trisiloxane, dimethicone, dimethicone/peg-10/15 crosspolymer, dioctanoyl isosorbide, shea butter, c12-15 alkyl benzoate, octocrylene, tetrahexyldecyl ascorbate, avobenzone, homosalate, dicaprylyl carbonate, ubiquinone, squalane, polyacrylate-15, polyacrylate-17, a lycium barbarum fruit extract, a panax gingseng root extract, phenoxyethanol, ammonium acryloyldimethyltaurate/vp copolymer, bisabolol, emulsifying wax NF (e.g., PolaWax P), sodium polyacrylate, a camellia oleifera (green tea) leaf extract, or combinations thereof.

The skin care composition provided herein can be formulated into appropriate types of vehicles for application to a mammal. Examples of vehicles that can be used as described herein include, without limitation, emulsions (e.g., oil-in-water, water-in-oil, silicone-in-water, water-in-silicone, water-in-oil-in-water, oil-in-water, oil-in-water-in-oil, oil-in-water-in-silicone, etc.), creams, lotions, solutions (both aqueous and hydro-alcoholic), anhydrous bases (such as lipsticks and powders), gels, ointments, pastes, milks, liquids, aerosols, solid forms, or eye jellies.

The skin care composition provided herein can be made by adding one or more of any of the ingredients disclosed herein together into a mixture. The mixture can be heated to an elevated temperature of about 90° C. (e.g., about 85° C., about 80° C., about 75° C., e.g., about 70° C., about 65° C., about 60° C., about 55° C., or about 50° C.). In some cases, the mixture can be heated to an elevated temperature ranging from about 50° C. to about 90° C. (e.g., from about 60° C. to about 80° C., about 65° C. to about 75° C., about 65° C. to about 70° C., about 50° C. to about 70° C., about 50° C. to about 65° C., or about 60° C. to about 65° C.). In some cases, one or more additional ingredients disclosed herein can be added to the mixture at the elevated temperature. In some cases, the one or more additional ingredients can be added to the mixture when the mixture is reduced from the elevated temperature to a lower temperature ranging from about 30° C. to about 60° C. (e.g., about 30° C. to about 55° C., about 30° C. to about 50° C., about 30° C. to about 45° C., 30° C. to about 40° C., 35° C. to about 60° C., 40° C. to about 60° C., about 45° C. to about 60° C., 50° C. to about 60° C., 55° C. to about 60° C., about 30° C. to about 45° C., 30° C. to about 40° C., about 35° C. to about 60° C., 40° C. to about 60° C., or about 50° C. to about 60° C.). In some cases, the one or more additional ingredients can be added to the mixture while applying shear forces to the mixture (e.g., apply shear mixing) to uniformly disperse the ingredients into the mixture. The mixture can be cooled to room temperature once the ingredients have been added.

The invention will be further described in the following examples, which do not limit the scope of the invention described in the claims.

### EXAMPLES

### Example 1

### An Exemplary Skin Care Composition

A skin care composition can be prepared with the ingredients shown in Table 1 below.

TABLE 1
Skin Care Composition for a Toner

| Ingredient | W/W % Range |
|---|---|
| Sapindus Mukorossi Extract | 0.01-0.5 |
| Allantoin | 0.05-1 |
| Carnosine | 0.1-2 |
| Plant Extract Blend Composed of | 0.1-10 |
| Bioflavonoids, Brassica oleracea ver. | |
| italica extracts, and Aloe barbadensis | |
| leaf extracts. | |

### Example 2

### An Exemplary Skin Care Composition

A skin care composition can be prepared with the ingredients shown in Table 2 below.

TABLE 2
Skin Care Composition for a Toner

| Ingredient | W/W % Range |
|---|---|
| Sapindus Mukorossi Extract | 0.001-0.5 |
| Allantoin | 0.05-1 |
| Carnosine | 0.05-2 |
| Plant Extract Blend Composed of | 0.1-10 |
| Bioflavonoids, Brassica oleracea ver. | |
| italica extracts, and Aloe barbadensis | |
| leaf extracts. | |

### Example 3

### An Exemplary Skin Care Composition

A skin care composition can be prepared with the ingredients shown in Table 3 below.

TABLE 3
Skin Care Composition for a Lotion

| Ingredient | W/W % Range |
|---|---|
| Sapindus Mukorossi Extract | 0.001-3 |
| Allantoin | 0.01-3 |
| Carnosine | 0.01-3 |

### Example 4

### An Exemplary Skin Care Composition

A skin care composition can be prepared with the ingredients shown in Table 4 below.

TABLE 4
Skin Care Composition for a Lotion

| Ingredient | W/W % Range |
|---|---|
| Sapindus Mukorossi Extract | 0.001-2 |
| Allantoin | 0.01-5 |
| Carnosine | 0.01-5 |

### Example 5

### An Exemplary Skin Care Composition

A skin care composition was prepared with the ingredients shown in Table 5 below, along with water and additional optional ingredients.

TABLE 5
Skin Care Composition for a Toner

W/W % Range

| Ingredient | Low | High |
|---|---|---|
| Carnosine | 0.1 | 2 |
| Allantoin | 0.05 | 1 |
| Sapindus Mukurossi Fruit Extract | 0.01 | 0.5 |

### Example 6

### An Exemplary Skin Care Composition

A skin care composition was prepared with the ingredients shown in Table 6 below, along with water and additional optional ingredients.

TABLE 6
Skin Care Composition for a Toner

W/W % Range

| Ingredient | Low | High |
|---|---|---|
| Carnosine | 0.05 | 2 |
| Allantoin | 0.05 | 1 |
| Sapindus Mukurossi Fruit Extract | 0.001 | 0.5 |

### Example 7

### An Exemplary Skin Care Composition

A skin care composition was prepared with the ingredients shown in Table 7 below, along with water and additional optional ingredients.

TABLE 7
Skin Care Composition for a Lotion

W/W % Range

| Ingredient | Low | High |
|---|---|---|
| Carnosine | 0.01 | 3 |
| Allantoin | 0.01 | 3 |
| Sapindus Mukurossi Fruit Extract | 0.001 | 3 |

### Example 8

### An Exemplary Skin Care Composition

A skin care composition was prepared with the ingredients shown in Table 8 below, along with water and additional optional ingredients.

TABLE 8
Skin Care Composition for a Lotion

W/W % Range

| Ingredient | Low | High |
|---|---|---|
| Carnosine | 0.01 | 5 |
| Allantoin | 0.01 | 5 |
| Sapindus Mukurossi Fruit Extract | 0.001 | 2 |

### Example 9

### An Exemplary Skin Care Composition

A skin care composition can be prepared with the ingredients shown in Table 9 below.

TABLE 9
Skin Care Composition for a Toner

| Ingredient | W/W % Range |
|---|---|
| Sapindus Mukorossi Extract | 0.01-0.5 |
| Allantoin | 0.05-1 |
| Plankton Extract and Water | 0.1-5 |
| Carnosine | 0.1-2 |
| Plant Extract Blend Composed of | 0.1-10 |
| Bioflavonoids, Brassica oleracea ver. | |
| italica extracts, and Aloe barbadensis | |
| leaf extracts. | |

### Example 10

### An Exemplary Skin Care Composition

A skin care composition can be prepared with the ingredients shown in Table 10 below.

TABLE 10
Skin Care Composition for a Toner

| Ingredient | W/W % Range |
|---|---|
| Sapindus Mukorossi Extract | 0.001-0.5 |
| Allantoin | 0.05-1 |
| Plankton Extract and Water | 0.01-5 |
| Carnosine | 0.05-2 |
| Plant Extract Blend Composed of | 0.1-10 |
| Bioflavonoids, Brassica oleracea ver. | |
| italica extracts, and Aloe barbadensis | |
| leaf extracts. | |

### Example 11

### An Exemplary Skin Care Composition

A skin care composition can be prepared with the ingredients shown in Table 11 below.

TABLE 11
Skin Care Composition for a Cleanser

| Ingredient | W/W % Range |
|---|---|
| Carnosine | 0.01-5 |
| Plant Extract Blend Composed of | 0.01-5 |
| Bioflavonoids, Brassica oleracea ver. | |
| italica extracts, and Aloe barbadensis | |
| leaf extracts. | |

### Example 12

### An Exemplary Skin Care Composition

A skin care composition can be prepared with the ingredients shown in Table 12 below.

TABLE 12
Skin Care Composition for a Cleanser

| Ingredient | W/W % Range |
|---|---|
| Allantoin | 0.01-5 |
| Carnosine | 0.01-5 |
| Plankton Extract and Water | 0.01-5 |

### Example 13

### An Exemplary Skin Care Composition

A skin care composition can be prepared with the ingredients shown in Table 13 below.

TABLE 13
Skin Care Composition for a Lotion

| Ingredient | W/W % Range |
|---|---|
| Sapindus Mukorossi Extract | 0.001-3 |
| Allantoin | 0.01-3 |
| Plankton Extract and Water | 0.01-5 |
| Carnosine | 0.01-3 |

### Example 14

### An Exemplary Skin Care Composition

A skin care composition can be prepared with the ingredients shown in Table 14 below.

TABLE 14
Skin Care Composition for a Lotion

| Ingredient | W/W % Range |
|---|---|
| Sapindus Mukorossi Extract | 0.001-2 |
| Allantoin | 0.01-5 |
| Plankton Extract and Water | 0.01-5 |
| Carnosine | 0.01-5 |

### Example 15

### An Exemplary Skin Care Composition

A skin care composition was prepared with the ingredients shown in Table 15 below, along with water and additional optional ingredients.

TABLE 15
Skin Care Composition for a Toner

W/W % Range

| Ingredient | Low | High |
|---|---|---|
| Plankton Extract and water | 0.1 | 5 |
| Carnosine | 0.1 | 2 |
| Allantoin | 0.05 | 1 |
| Sapindus Mukurossi Fruit Extract | 0.01 | 0.5 |

### Example 16

### An Exemplary Skin Care Composition

A skin care composition was prepared with the ingredients shown in Table 16 below, along with water and additional optional ingredients.

TABLE 16
Skin Care Composition for a Toner

W/W % Range

| Ingredient | Low | High |
|---|---|---|
| Plankton Extract and water | 0.01 | 5 |
| Carnosine | 0.05 | 2 |
| Allantoin | 0.05 | 1 |
| Sapindus Mukurossi Fruit Extract | 0.001 | 0.5 |

### Example 17

### An Exemplary Skin Care Composition

A skin care composition was prepared with the ingredients shown in Table 17 below, along with water and additional optional ingredients.

TABLE 17
Skin Care Composition for a Cleanser

W/W % Range

| Ingredient | Low | High |
|---|---|---|
| Carnosine | 0.01 | 5 |

### Example 18

### An Exemplary Skin Care Composition

A skin care composition was prepared with the ingredients shown in Table 18 below, along with water and additional optional ingredients.

TABLE 18
Skin Care Composition for a Cleanser

W/W % Range

| Ingredient | Low | High |
|---|---|---|
| Carnosine | 0.01 | 5 |
| Allantoin | 0.01 | 5 |
| Plankton Extract and Water | 0.01 | 5 |

### Example 19

### An Exemplary Skin Care Composition

A skin care composition was prepared with the ingredients shown in Table 19 below, along with water and additional optional ingredients.

TABLE 19
Skin Care Composition for a Lotion

W/W % Range

| Ingredient INCI Name | Low | High |
|---|---|---|
| Carnosine | 0.01 | 3 |
| Allantoin | 0.01 | 3 |
| Sapindus Mukurossi Fruit Extract | 0.001 | 3 |
| Plankton Extract and Water | 0.01 | 5 |

### Example 20

### An Exemplary Skin Care Composition

A skin care composition was prepared with the ingredients shown in Table 20 below, along with water and additional optional ingredients.

TABLE 20
Skin Care Composition for a Lotion

W/W % Range

| Ingredient | Low | High |
|---|---|---|
| Carnosine | 0.01 | 5 |
| Allantoin | 0.01 | 5 |
| Sapindus Mukurossi Fruit Extract | 0.001 | 2 |
| Plankton Extract and water | 0.01 | 5 |

### Other Embodiments

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

What is claimed is:

1. A skin care composition comprising:
(a) a Sapindus mukorossi extract in an amount from about 0.001% to about 0.5% by weight of said composition;
(b) allantoin in an amount from about 0.05% to about 1% by weight of said composition; and
(c) carnosine from about 0.1% to about 2% by weight of said composition.

2. The composition of claim 1, wherein said composition further comprises a plant extract blend in an amount from about 0.1% to about 10% by weight of said composition, wherein said plant extract blend comprises a bioflavonoid, a Brassica oleracea ver. italica extract, and an Aloe barbadensis leaf extract.

3. The composition of claim 1, wherein said composition is a topical skin care formulation.

4. The composition of claim 1, wherein application of said composition to human skin causes said skin to become hydrated.

5. The composition of claim 1, wherein application of said composition to human skin causes said skin to be cleansed of makeup, dirt and excess oil.

6. A skin care composition comprising:
(a) a Sapindus mukorossi extract in an amount from about 0.001% to about 0.5% by weight of said composition;
(b) allantoin in an amount from about 0.05% to about 1% by weight of said composition;
(c) a plankton extract from about 0.1% to about 5% by weight of said composition; and
(d) carnosine from about 0.1% to about 2% by weight of said composition.

7. The composition of claim 6, wherein said composition further comprises a plant extract blend in an amount from about 0.1% to about 10% by weight of said composition, wherein said plant extract blend comprises a bioflavonoid, a Brassica oleracea ver. italica extract, and an Aloe barbadensis leaf extract.

8. The composition of claim 6, wherein said composition is a topical skin care formulation.

9. The composition of claim 6, wherein application of said composition to human skin causes said skin to become hydrated.

10. The composition of claim 6, wherein application of said composition to human skin causes said skin to be cleansed of makeup, dirt and excess oil.
